(19) United States
(12) Patent Application Publication
(10) Pub. No.: US 2005/0021068 A1
(43) Pub. Date: Jan. 27, 2005

(54) Method of stripping diseased blood vessels from the human body

(76) Inventors: John C. Opie, Scottsdale, AZ (US); Stephen J. Joyce, Phoenix, AZ (US)

Correspondence Address: SQUIRE, SANDERS & DEMPSEY L.L.P.;Two Renaissance Square, Suite 2700, 40 North Central Avenue, Phoenix, AZ, 85004-4498, US

(21) Appl. No.: 10/868,746

(22) Filed: Jun. 14, 2004

Related U.S. Application Data
(60) Provisional application No. 60/478,474, filed on Jun. 12, 2003.

(51) Int. Cl. A61B 17/32

(52) U.S. Cl. 606/190

(57) ABSTRACT

A surgical device and method for using it to strip blood vessels, such as varicose veins, or other tubular structures from the body utilizing an endovenous cable that is passed through the vein section to be removed. Once the endovenous cable reaches the end of the section of blood vessel to be stripped, a head is attached to the endovenous cable. The head may have at its leading edge, rounded, streamlined shoulders, a leading edge and a central cavity to retain the end of the vein. On the trailing end of the stripper head are one or more structures capable of receiving and retaining a surgical drain. As the vein is tripped the drain is automatically pulled into the body. The drain will lie in the leg precisely where the vein had been, and the limit the common complication of retained post surgical bleeding.

### RELATED APPLICATIONS

This application claims priority to U.S. Provisional Application Ser. No. 60/478,474, filed Jun. 12, 2003. The disclosure of U.S. application Ser. No. ______, filed Jun. 7, 2004, entitled SURGICAL DRAINS to John C. Opie, Stephen J. Joyce, and Thomas Izdebski, is hereby incorporated by reference.

### FIELD OF THE INVENTION

This invention relates to surgical instruments and their use. Specifically, the invention relates to surgical instruments used for removing blood vessels, particularly diseased and unsightly varicose veins, or other tubular structures, from the body.

### BACKGROUND OF THE INVENTION

Varicose veins have a multitude of undesirable features. It is estimated that about 80 million people in the USA at any one time suffer from some form of varicose venous disease. Significantly diseased varicose veins cause considerable pain, especially while the afflicted person is standing, and they are also unsightly as they bulge while the person is standing, which is due to the valvular incompetence of the vein. Varicose veins are a condition for which many patients annually seek a surgical remedy. It is not at all uncommon for a patient to enter a physician's office with very painful superficial thrombophlebitis, and sometimes it is possible for pulmonary emboli to occur from above-knee greater saphenous vein (GSV) thromboses. It is also possible with very superficial, high-pressure, large, varicose veins that a patient might suffer an inconsequential skin abrasion (usually near the shin), and then the vein will open and could threaten a patient's life from exanguination, for example, while the patient is asleep.

Long-standing incompetent varicose veins may cause a steady buildup of iron in the subcutaneous fat layers and skin of the lower leg, resulting in permanent staining of the tissues, known as hemosiderosis. As this condition progresses the subcutaneous tissues become woody and hard and take on a rust colored appearance. This can result in substantial local pain, embarrassment and unsightly rust-colored skin depressions from fat necrosis. As the circumstances advance, the skin, especially over and above the medial malleolus and the anteromedial lower shin, is apt to break down and infection is prone to set in. Advanced venous stasis ulcers can be difficult to resolve. Varicose veins may be secondary to sapheno-femoral valve regurgitation, common femoral valve regurgitation or to incompetent perforators.

It is in the best interests of the patient to prevent progression from early varicose veins to advanced complicated varicose veins and stasis ulcer formation. Provided that it is established that the patient has sapheno-femoral valve incompetence, and consequent GSV regurgitation, documented, for example, by venous color flow, duplex Doppler studies performed with and without a Valsalver maneuver, then a long, or GSV, stripping is a good procedure that can prevent most of the advanced complications from developing over time. As used herein, “stripping” means removal of all or part of the GSV. As described below, removing, or stripping, the GSV means to literally pull it out of the body.

The known method for removal of the greater saphenous vein (“GSV”) typically involves exposing it with an incision in the groin area, and delivering the GSV at its junction with the common femoral vein, just below the inguinal ligament. Once delivered, the GSV is ligated in continuity and is then exposed in front of the medial malleolus at the ankle, and the GSV at the ankle is also ligated in continuity and divided. A flexible plastic stripper endovenous cable is then passed through the GSV and is variably threaded to the groin section of the GSV. On occasions it is not possible to pass the stripper endovenous cable through the GSV due to knots and snarls within the GSV. Secondary incisions then become necessary and the process is repeated, thus removing sections of the GSV at a time. Once at the groin, a ligature is passed around the GSV and loosely tied to prevent excessive blood loss. At this time the GSV is now divided and the terminal expansion of the stripper endovenous cable is passed out of the vein. At this time the second ligature is firmly tied around the stripper endovenous cable. A stripper head is now attached to the stripper endovenous cable and the head is pulled through the leg to secure the endovenous cable in the GSV as the GSV is stripped.

In the known method of Babcock/Meyer, the stripper head is similar to a mushroom in shape and comes in three sizes, large, medium and small. Typically, the medium size is selected for removal of the GSV. The stripper head is fitted into the groin incision and the vein is then serially avulsed from the leg from a longitudinal pull from the ankle incision where the endovenous cable is attached to some form of a handle. The GSV bunches up against the leading surface of the stripper bead. In order to reduce bleeding as the vein is stripped the leg may be either compressed with a firm ACE bandage around the leg before the strip is performed or local pressure can be applied as the stripper head passes down the leg. Once that is done the two or more incisions are closed with sutures and the patient is sent home after several recovery hours with an ACE bandage around the leg.

Three or four days later the patient usually visits the doctor's office and the ACE wrap is removed. More commonly than not, the leg has significant ecchymoses, cords of contained hematomas down the line where the veins were stripped and usually the patient has a painful, swollen leg. The bruising often takes several weeks to resolve as does the discoloration from the post-operative hemorrhage that usually occurs. Sometimes it is necessary to return the patient to the operating room to surgically release pressurized (and potentially painful) hematomas.

Prior art stripping techniques involved using rings to locate branches. These ringed instruments are passed blindly from above down under the skin and next to the vein from the groin incision. Once a substantial branch is located by observing pulling of the skin, a local incision is made and the branch may be tied.

Other prior art involves non-stripping procedures. Such procedures include radio frequency ablation, laser ablation and sclerotherapy. The former two procedures produce clots in the varicosed vein by heat, the latter by invoking clot formation with pro-coagulant chemicals. Each procedure has the potential to produce pulmonary embolism. The main problem with these techniques is centered around the continued existence of the varicosed vein. In all of these techniques there may be significant pain, risk of pulmonary embolism and recurrence or recannelization of the veins. Also, the vein remains in situ and can remain unsightly. A significant number of patients using these alternate techniques revert to traditional stripping technique of Babcock/Meyers.

Prior art techniques also involve inversion stripping. In this situation, the endovenous cable is passed as before through the vein but no stripper head is attached. Rather, the vein is simply tied to the endovenous cable before the expansion. The vein is now pulled out of the leg and inverts on itself as it does so; it is literally peeled out of the body. The main problem with this technique is that as the vein inverts, the bulky, large tubular part of the vein, which lies in the groin and thigh must fit inside the smaller, less bulky part of the vein in the calf area. Sometimes, it is impossible for the inversion technique to continue and the vein is simply torn apart and the stripping stops. That immediately results in bleeding and necessitates an additional incision to locate the unstripped vein portion, which must be retrieved and stripped. Thus while invagination stripping is less traumatic that the Babcock/Meyers method, it is less effective in facilitating successful stripping of varicose veins. For this reason the Babcock/Meyers stripping procedure has become the procedure of common use, but the main problems with the Babcock/Meyers procedure are (1) trauma, and (2) postoperative bleeding and its complications.

The traumatic component of the Babcock/Meyers technique can be easily understood by understanding how the stripper head is designed. It is similar to a toadstool shape with a cone-like trailing end but a flat or blunt leading end that must be pulled through the patient's tissues. Pulling such a blunt ended instrument through the patient's leg from the groin to the ankle, although the head holds the crumpling vein, remains quite traumatic and considerable force is required to pull the flat cone down the full length of the leg. As larger stripper heads are selected, the pulling force required rises substantially. The reverse is obviously also true. The smaller head, however, runs a risk that the vein will flow over the head and not be stripped.

At least these two concerns, trauma and hemorrhage, of the simple Babcock/Meyers technique should be addressed and solved.

### SUMMARY OF THE INVENTION

The present invention addresses the above problems by using, among other things, a smaller and more streamlined stripper head without significant risk of the vein flowing over the head and into the body during stripping. This smaller stripper head substantially diminishes the trauma associated with removing the vein. As used herein with respect to the invention, “vein” includes all tubular body structures, unless a specific structure, such as the GSV, is identified.

The present invention improves upon the prior art by providing relatively simple surgical instruments for stripping veins from a body.

The preferred embodiment of the invention comprises three main parts: an endovenous cable for placement into the vein, a stripper head for helping to retain the endovenous cable in the vein and preventing the vein from backing up into the body as it is stripped, and a handle to pull the endovenous cable and stripper head through the body.

The endovenous cable is preferably a solid plastic rod, although any suitable endovenous cable for passing through the vein and assisting in stripping the vein may be used. The endovenous cable has a first end and a second end and each end preferably has an expansion in the form of a flat, oval-shaped section.

The stripper head is preferably a low-profile, streamlined structure and has a leading edge and a smooth outer surface. Rounded edges connect the leading edge to the outer surface. The stripper head also has an inner cavity for receiving part of the vein as it is stripped, and a slot for attaching the stripper head to an expansion of the endovenous cable.

The handle can be any suitable structure for pulling the endovenous cable and stripper head out of the body, thereby stripping the vein. Preferably, the handle is plastic and has a central slot for receiving an expansion of the endovenous cable.

Also disclosed herein is a method of using the device and a novel endovenous cable, stripper head and handles that may be used to practice the invention.

### BRIEF DESCRIPTION OF THE DRAWINGS

FIG. 1 is a plan view of an endovenous cable of the present invention with expansions on either end.

FIG. 2 is a side, partial sectional view of the preferred embodiment of the stripper head of the invention.

FIG. 3 is a plan view of a plastic handle applied to the endovenous cable sufficient to facilitate the application of force to pull out a vein.

FIG. 4 is a plan view of an alternate handle design.

FIG. 5 is a side view of the handle of FIG. 4.

### DETAILED DESCRIPTION OF THE PREFERRED EMBODIMENTS

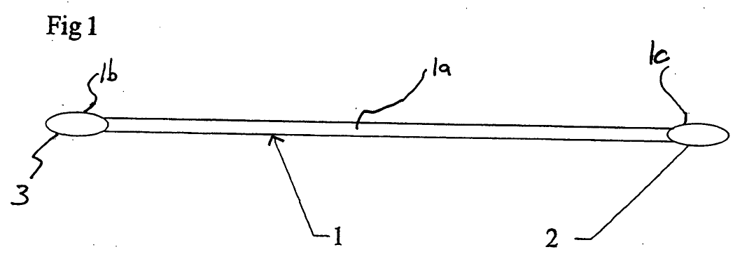

Referring now to the drawings where the purpose is to describe preferred embodiments of the invention and not to limit same, FIG. 1 shows a preferred embodiment of an endovenous cable 1 of the present invention. Endovenous cable 1 preferably comprises an elongate, flexible, solid, plastic (most preferably polycarbonate, although any suitable material may be used) rod approximately 30% longer than the average human leg, and most preferably about 100 cm in length. Endovenous cable 1 preferably has a cylindrical center section 1a, a thickness of about 1 mm, a first end 1b, and a second end 1c. Endovenous cable 1 is preferably strong enough to resist breaking when pulled by an adult male to strip a vein, and is preferably flexible enough to be threaded through a tortuous varicose vein with relatively gently applied distal force.

At each end 1b, 1c, respectively, of endovenous cable 1 are expansions 2, 3. Expansions 2, 3 take the general shape of an oval in plan view, and are preferably egg-shaped when viewed in three dimensions. Each expansion 2, 3 is about 3 mm+/−1 mm wide at the widest point. Expansions 2, 3 are about 0.5 cm long and are designed to attach to either the stripper head or the handle by being received in a suitable receptacle or slot. Each expansion 2, 3 preferably has the same dimensions so that they are interchangeable, although they may be of different shapes or sizes. Once in place in a receptacle or slot in either the handle or the stripper head, they cannot be easily removed by longitudinal force. They could possibly be removed, however, by slipping the expansion 2 or 3 laterally with respect to the slot in either the handle or the stripper head, if removal is a desired feature.

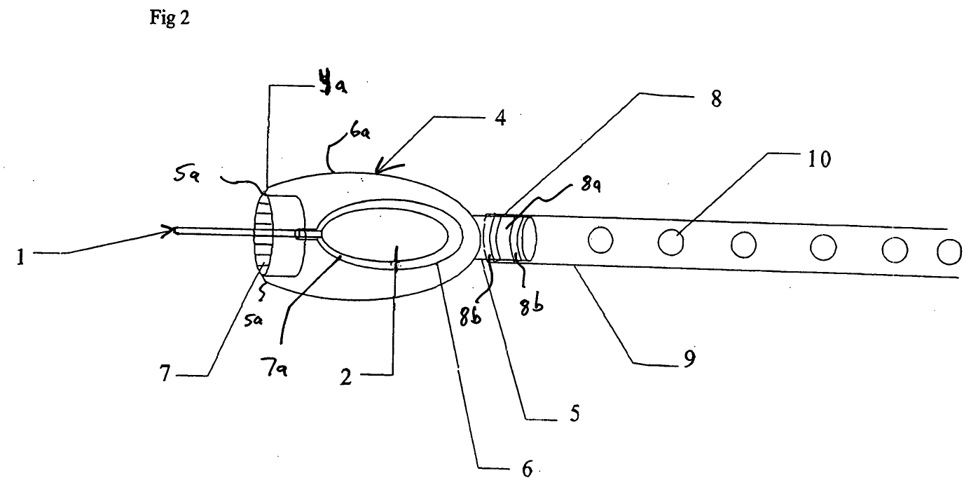

Referring now to FIG. 2, stripper head 4 has leading surface (or leading edge) 4a and an internal cavity 7 for receiving part of the vein during stripping and preventing the vein from entering the cavity formed in the body during the stripping process. Any suitable structure for this purpose may be used, although cavity 7 is preferred. Once the vein is received in internal cavity 7 it cannot easily pass over the stripper head 4 so stripper head 4 does not need to be as large as with the afore-mentioned Babcock/Meyers procedure. Also, because this in not an invagination technique, the vein will not tear apart with longitudinal force as it enters the calf area.

Stripper head 4 has a lateral slot 6 that will accept an expansion 2 or 3 of endovenous cable 1 and retain the expansion in place inside stripper head 4. Any suitable structure for attaching stripper head 4 to endovenous cable 1 may be used, however. Expansion 2 or 3 and slot 6 are simply an easy way to make the attachment.

Leading edge 4a of stripper head 4 has a rounded edge 5a and a preferably smooth outer surface 6a. Edge 5a makes it much less traumatic as stripper head 4 is pulled through a patient's leg tissues. Much less force will be required due to the streamlined leading edge and smaller size of stripper head 4. This is possible in part because of internal cavity 7 designed to receive the vein thus preventing it from flowing over the smaller profile stripper head of the invention.

On the trailing end 5 of stripper head 4 is a cylindrical projection 8. Projection 8 is a structure for attaching a surgical drain to stripper head 4 and any suitable structure may be used. As shown, trailing end 8 is cylindrical, has an outer surface 8a and one or more raised circumfrential chevrons 8b. Projection 8 is designed to receive and temporarily retain a drain 9 of sufficient length to extend down (preferably) the entire length of the leg if the GSV is being stripped. Thus, in the preferred embodiment, as stripper head 4 is pulled down the leg stripping a vein, stripper head 4 will also pull a surgical drain, such as drain 9, into the precise space in the body from which the vein is being stripped. Previous to this invention, such automatic drain placement was not possible. Once placed in position, drain 9 is then simply exterorized at a convenient location and the ankle and groin incisions are closed in the routine fashion. Such a drain makes subcutaneous hematomas, extensive bruising and ecchymoses much less unlikely.

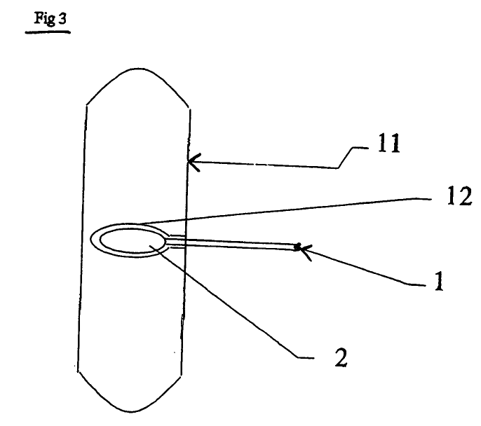

FIG. 3 shows a handle 11 made of suitable plastic (although any suitable material may be used) to be pulled and apply enough force to pull endovenous cable 1 and stripper head 4 through the body. Handle 11 includes a central cavity 12 designed to accept one of expansions 2 or 3 on endovenous cable 1 in a sliding manner such that when longitudinal force is applied to the endovenous cable 1 the expansion will not slip out of the handle. However, as previously described, any suitable structures for attaching handle 11 to endovenous cable 1 may be used. Handle 11 (and all handles according to the invention) is of suitable size, shape, strength and thickness to be held and pulled, without significant deformation, while a vein is being stripped.

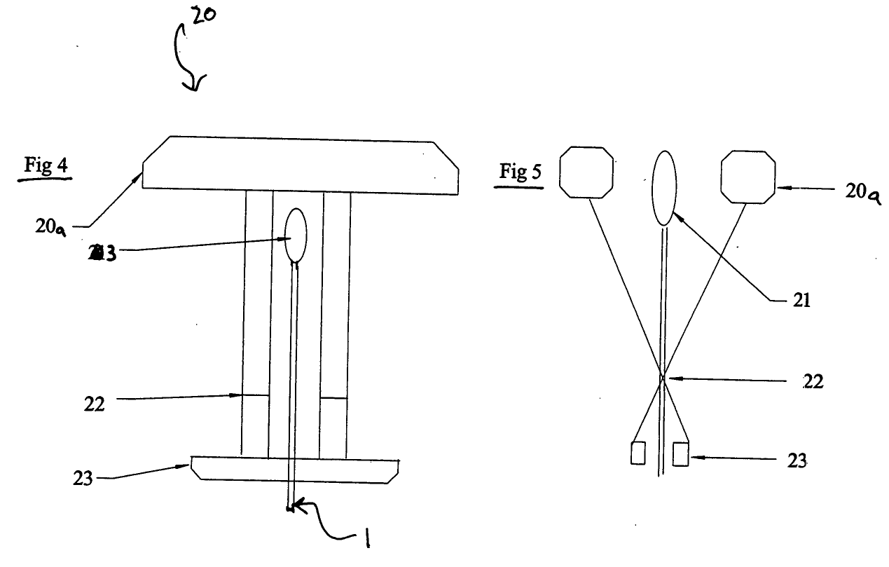

FIGS. 4 and 5 show an alternate handle 20 that may be used with the invention. The advantage of this style of handle is that it allows the handle to be moved without restriction up or down endovenous cable 1 but grab endovenous cable 1 at any point with sufficient force to strip a vein according to the teachings of the invention. Handle 20 has a squeezable plastic handle 20a (also seen in FIG. 5) designed to be compressed by the pulling hand. Such compression on handle 20a transmits a multiplied force to plastic gripper pads 23, which grab the stripper endovenous cable at any movable point. There is a fulcrum point 22 of a pair arms made of plastic or metal further toward the grippers so that a magnified force is available to the grippers when squeezing the handles 20.

Prior art handles only faster to an end of the endovenous cable. This means as the stripper head is pulled past (i.e., below) the knee when the GSV is removed, the endovenous cable outside of the body is now quite long and is well past the foot. As the stripping continues the handle gets even further away from the operative site. This can be undesirable and potentially dangerous. The application of force with this system is poorly controlled with respect to proximity to the patient. The advantage a moveable handle provides is that a surgeon doing the stripping, for the first time, can now stand close to the distal incision, which is at the ankle for GSV removal, during the entire procedure. First, a segment of vein in the thigh is avulsed. The surgeon can then release the grip on the handles and slide the handle up the endovenous cable and thus closer to the patient's ankle and then continue pulling. This provides better and safer control.

The vein stripping instrument of the present invention thus provides multiple functions that have not before been available. The present invention provides one or more of the following: a one size fits all stripper head (although different sizes may be used) with a vein trapping feature (the internal cavity), reduced tissue trauma (due to the size and sloping streamlined edges), and preferably provides a solution for the problem of postoperative hemorrhage (the ability to attach a trailing drain to the device, preferably to the stripper head to drain any post operative hemorrhage) with or without an ACE bandage.

Having now described preferred embodiments of the invention, modifications and variations to the present invention may be made by those skilled in the art. The invention is thus not limited to the preferred embodiments, but is instead set forth in the following claims and legal equivalents thereof.

1. A device for stripping veins or other tubular structures from a body, the device comprising:
a) an endovenous cable;
b) a stripper head having a leading edge and an internal cavity, the stripper head attachable to the endovenous cable;
c) a handle attachable to the endovenous cable and capable of allowing a user to apply force to the endovenous cable; and
d) a structure for connecting a surgical drain to the device.

2. The device of claim 1 wherein the stripper head is comprised of steel.

3. The device of claim 1 wherein the stripper head has an outer wall and rounded edges leading from the leading edge to the outer wall.

4. The device of claim 1 wherein the internal cavity is downstream of the leading edge when the device is in use.

5. The device of claim 1 wherein the stripper head has a maximum width of between 7 mm and 10 mm.

6. The device of claim 1 wherein the endovenous cable is a solid polycarbonate rod.

7. The device of claim 1 wherein the endovenous cable is about 100 cm in length.

8. The device of claim 1 wherein the endovenous cable has a first end and a second end and at least one of the ends is expanded.

9. The device of claim 8 wherein both ends are expanded.

10. The device of claim 8 wherein the at least one expanded end is egg-shaped.

11. The device of claim 10 wherein the at least one expanded end is oval in plan view.

12. The device of claim 8 wherein the stripper head attaches to the endovenous cable by the at least one expanded end being received in a slot in the stripper head.

13. The device of claim 12 wherein the slot is in the internal cavity.

14. The device of claim 8 wherein the handle is attached to the endovenous cable by attaching to the at least one expanded end.

15. The device of claim 1 wherein the handle is plastic.

16. The device of claim 1 wherein the handle has a central slot for attaching to an expanded end of the endovenous cable.

17. The handle of claim 1 that may be moved along the endovenous cable and that may apply pulling force at any point it is placed on the endovenous cable.

18. A handle for a medical device used to strip veins or other tubular structures from a body, the handle comprising:
a) a generally planar body and thickness suitable for grasping with both human hands; and
b) a slot for receiving an expansion of an endovenous cable.

19. The handle of claim 18 that is comprised of plastic.

20. The handle of claim 18 that has a central slot for attaching to an expansion of the endovenous cable.

21. The handle of claim 18 that may be moved along the endovenous cable and that may apply pulling force at any point it is placed on the endovenous cable.

22. An endovenous cable for a medical device used to strip veins or other tubular structures from a body, the endovenous cable comprising:
a) a cylindrical body;
b) a first end;
c) a second end;
d) a first expansion formed at the first end for connecting to a slot in a stripper head, and a second expansion formed at the second end, the second expansion for connecting to a handle.

23. The endovenous cable of claim 22 that is comprised of plastic.

24. The endovenous cable of claim 22 that is 100 cm in length.

25. A stripper head for a medical device used to strip veins or other tubular structures for a body, the stripper head comprising a leading edge and an internal cavity downstream of the leading edge.

26. The stripper head of claim 25 that has a maximum width of between 7 cm and 10 cm.

27. The stripper head of claim 25 that has an outer surface and rounded edges leading to the rounded surface.

28. The stripper head of claim 25 that has a slot for receiving an expansion of an endovenous cable.

29. The stripper head of claim 25 that includes a structure for securing a surgical drain.

30. The stripper head of claim 29 wherein the structure comprises a cylindrical trailing edge.

31. The stripper head of claim 29 wherein the structure further comprises one or more raised circumferential chevrons on the trailing edge.

32. A method of removing a vein from a body, the method comprising the steps of:
a) making a first incision in the body to access a first section of the vein and making a second incision in the body to access a second section of the vein;
b) extending an endovenous cable having a first end and a second end into the vein near the first incision, through the vein, and out of the vein near the second incision;
c) attaching a stripper head having a leading edge and an internal cavity to the first end of the endovenous cable;
d) attaching a handle to the second end of the endovenous cable;
e) pulling the handle and pulling the vein, endovenous cable and stripper head through the body and out of the second incision.
